(19) United States
(12) Patent Application Publication
(10) Pub. No.: US 2005/0256361 A1
(43) Pub. Date: Nov. 17, 2005

(54) Implant inserting device

(76) Inventors: Christian Mathieu, Le Brassus (CH); Frederic Heimgartner, Martigny (CH); Kamel Besseghir, Lausanne (CH)

Correspondence Address: NIXON & VANDERHYE, PC, 901 NORTH GLEBE ROAD, 11TH FLOOR, ARLINGTON, VA, 22203, US

(21) Appl. No.: 10/519,492

(22) PCT Filed: Jul. 2, 2003
(86) PCT No.: PCT/CH03/00437

§ 371 Date: Jun. 16, 2005

(51) Int. Cl. A61N 5/00; A61M 36/00; A61M 31/00

(52) U.S. Cl. 600/7; 604/60; 604/506

(57) ABSTRACT

The invention concerns a device (1) for inserting implants (10) in the form of a cylinder of small diameter comprising gripping means (2), a trocar (3) fixed through its proximal end (4) to the gripping means (2), and a push-rod (5) mounted sliding through the trocar (3) and the gripping means (2). The invention is characterized in that the gripping means (2) include a rotating element (6) defining an axis of rotation (7) parallel to the axis of the trocar (8) and comprising a plurality of tubular elements (9) arranged around said axis of rotation (7) and mounted so as to be aligned successively with the trocar (3), each tubular element (9) being adapted to contain at least two implants (10) arranged one behind the other.

The invention relates to a device for inserting implants in the form of cylinders of small diameter. It relates more particularly to a device permitting subcutaneous insertion of several implants in succession.

Successive insertion of implants has a number of applications in the medical field, especially in the treatment of chronic diseases, such as acromegaly, when the patient receives a series of implants which are in most cases biodegradable and contain an active substance.

U.S. Pat. No. 4,086,914 describes a device for inserting a series of implants in succession. The device comprises a trocar and a push rod mounted so as to slide through the trocar. The implants are introduced into the device by means of a cartridge of tubular shape which contains a series of implants. The cartridge is initially introduced into a compartment situated in the continuation of the proximal end of the trocar. By means of the push rod, the series of implants is then moved in the direction of the distal end of the trocar. If, for a given treatment, the number of implants contained in the cartridge is insufficient, the empty cartridge is replaced by a full cartridge.

Replacing a cartridge is a delicate procedure. Several maneuvers are required, and this not only results in an increased duration of the treatment, but also causes the patient additional pain due to the movement of the trocar when the cartridge is being replaced.

The present invention aims in particular to remedy the aforementioned disadvantages.

It relates to a device for inserting implants in the form of cylinders of small diameter, comprising gripping means, a hollow needle (called a trocar hereinafter) fixed at its proximal end to the gripping means, and a push rod mounted so as to slide through the trocar and the gripping means, characterized in that the gripping means include a rotary element defining an axis of rotation parallel to the trocar axis and comprising a plurality of tubular elements arranged around said axis of rotation and mounted so as to be able to be aligned successively with the trocar, said rotary element forming an integral part of the gripping means and extending along most of the length of said gripping means, each tubular element being designed to contain at least one implant.

The tubular elements can be formed directly in the rotary element.

However, because of legal requirements associated with medical instruments, in particular for reasons relating to sterility of the device, and also to ensure optimal storage of the implants, it is desirable to use tubular elements which form a part distinct from the rotary element. This configuration affords the possibility of separating the implants from the insertion device prior to treatment.

In addition, if the treatment does not require the use of all the cartridges that can be loaded on the rotary element, it is possible to load only the required number of cartridges. This ensures that the patient is administered the exact dose for treatment of his or her disease. Moreover, in many cases the financial savings made in this way may be quite considerable, because the cost of an implant can be very high.

It should be noted that the rotary element can be loaded at the time of production, or by the practitioner prior to treatment.

An illustrative embodiment of the invention will be described below with reference to the following figures in which:

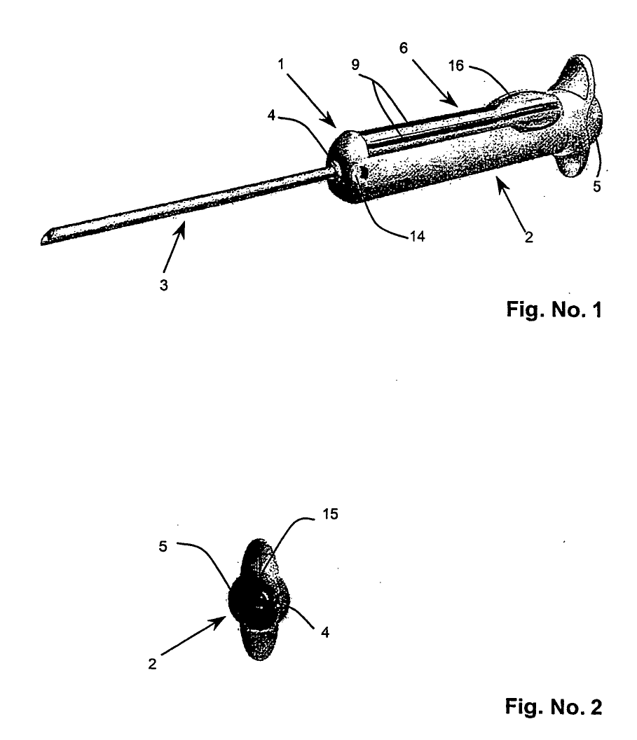

FIG. 1 shows the insertion device ready for use,

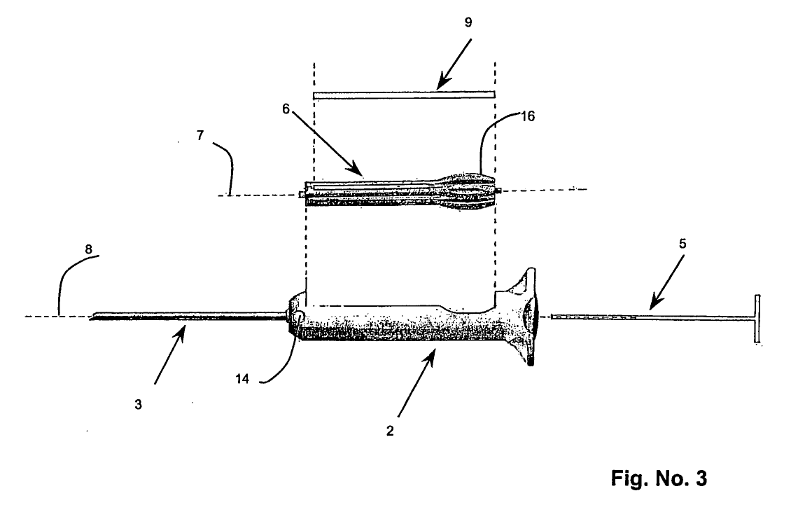

FIG. 2 shows a front view of the device,

FIG. 3 shows an exploded view of the device,

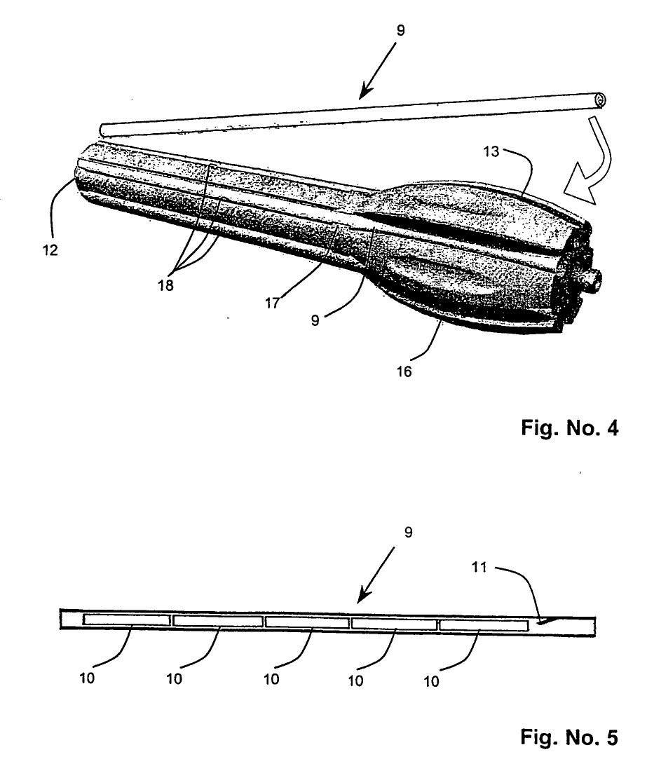

FIG. 4 depicts the barrel and several cartridges,

FIG. 5 shows a cartridge containing a series of five implants.

List of reference numbers used in the figures:
1 device for inserting implants
2 gripping element
3 trocar
4 proximal end of the trocar
5 push rod
6 barrel
7 axis of rotation of the barrel
8 main axis of the trocar
9 cartridge
10 implant
11 retention tongue
12 distal retention clip
13 groove
14 window
15 cross section of the gripping element
16 knurled wheel
17 proximal retention clip
18 compartment for cartridge

The device illustrated in FIGS. 1 and 3 comprises a trocar 3, and a gripping element 2 on which the proximal end 4 of the trocar 3 is fixed. A push rod 5 is mounted so as to slide through the trocar 3 and the gripping element 2. The external diameter of the push rod 5 is substantially equal to the internal diameter of the trocar 3.

The gripping element 2 comprises a rotary element 6, called a barrel hereinafter (see also FIG. 4), equipped at its proximal end with an enlargement forming a knurled wheel 16. The barrel 6 comprises several tubular elements 9, made of metal for example and called cartridges hereinafter, these being snap-fitted onto the body of the barrel 6. According to a preferred embodiment, five cartridges 9 are used. Each cartridge 9 contains a series of implants 10 arranged successively one behind the other. The choice of size of the implants is not limited. However, a length of between 0.5 and 2 cm and a diameter of 1.7 to 1.9 mm will generally be chosen. The axis of rotation 7 of the barrel 6 is parallel to the main axis of the device 1, in particular to the axis 8 of the trocar 3. The barrel 6 is designed in such a way as to permit successive alignment of the cartridges 9 with the trocar 3. The gripping element 2 additionally includes a window 14 through which it is possible to view the passage of the implants 10 as they move through the gripping element 2. Instead of the window 14, it is possible to provide another means of determining the position of the push rod 5, for example a graduation marked on the push rod 5.

As will be seen in particular from FIG. 4, the barrel 6 includes a series of compartments 18 in which the cartridges 9 are fitted. Once fitted, said cartridges 9 cannot be withdrawn, because of the presence of retention clips 12, 17 and the grooves 13 of the knurled wheel 16 at the bottom of which the cartridges 9 lodge. Single use of the cartridges 9 is thus ensured.

The gripping element 2 is preferably designed such that the barrel 6 is subsequently introduced into the gripping element 2 at its distal end.

FIG. 5 shows a cartridge 9 containing a series of five implants 10 arranged in succession. When the device 1 is at rest, rearward movement of the implants 10 is avoided by the presence of a flexible tongue 11 situated toward the proximal end of the cartridge 9. In addition, a similar system for retention of the implants 10 can be arranged toward the distal end of the cartridge 9.

The cross section 15 of the gripping element 2 is of oval shape or more generally flattened (see FIG. 2) so as to facilitate placing the device on the patient's skin and to reduce the pain which could be induced by a trocar forming a substantial angle with the surface of the skin.

According to a preferred embodiment (not shown), the device comprises means for retention of the barrel 6 which prevent withdrawal of the barrel once the latter has been placed on the gripping element 2. These means can comprise a retention tongue which breaks off if an attempt is made to remove the barrel.

The way in which the device works will be described below.

Before use, the device is preferably separated into different parts. The cartridges 9 containing the implants 10 are kept to one side, and the barrel 6 is not fixed to the gripping element 2.

To make the whole assembly compact, the push rod 15 can be introduced completely into the gripping element 2 and into the trocar 3.

In a first step, the cartridges 9 are loaded onto the barrel 6 by snap-fitting.

The barrel 6 is then fixed on the gripping element 2 as has been indicated above.

Before the barrel 6 is fixed on the gripping element 2, the push rod 5 can be withdrawn.

Alternatively, the push rod 5 remains in place. In this case, the barrel 6 contains a cartridge-free compartment (not shown) which takes up a position exactly on the push rod 5.

Once the barrel 6 is in place, the tip of the trocar 3 is introduced beneath the patient's skin. At this moment, the push rod 5 is preferably driven in so as to avoid a punch effect.

The push rod 5 is then withdrawn in order to allow a first full cartridge 9 to come into alignment with the trocar 3 by rotating the barrel 6 at the area of the knurled wheel 16.

It will be noted here that the barrel is preferably rotated in a non-continuous manner, in increments (snap-fitting), in such a way as to immediately align the cartridges 9 with the trocar 3.

The push rod 5 is then moved in the direction of the distal end of the trocar 3. In doing this, the flexible tongue 11 is pushed aside toward the outside of the cartridge 9.

When the implants 10 are situated toward the distal end of the trocar 3, the gripping element 2 is gradually withdrawn while the push rod 5 remains fixed in position so as to place the chain of implants 10 in the cavity formed by the trocar 3 beneath the skin.

Once the cartridge 9 has been emptied, this phase being observed through the window 14, the push rod 5 is withdrawn so as to permit renewed rotation of the barrel 6 in order to align a new cartridge 9 with the trocar 3. The procedure will be identical with the other cartridges 9.

1. A device (1) tar inserting implants (10) in the form of cylinders of small diameter, comprising gripping means (2), a trocar (3) fixed at its proximal end (4) to the gripping means (2), and a push rod (5) mounted so as to slide through the trocar (3) and the gripping means (2), characterized in that the gripping means (2) include a rotary element (6) defining an axis of rotation (7) parallel to the trocar axis (8) and comprising a plurality of tubular elements (9) arranged around said axis of rotation (7) and mounted so as to be able to be aligned successively with the trocar (3), said rotary element (6) forming an integral part of the gripping means (2) and extending along most of the length of said gripping means, each tubular element (9) being designed to contain at least one implant.

2. The device as claimed in claim 1, characterized in that each tubular element (9) forms a part distinct from the rest of the rotary element (6)

3. The device as claimed in claim 2, characterized in that each tubular element (9) can be inserted into the rotary element (6).

4. The device as claimed in claim 2, characterized in that it comprises means (12, 13) which prevent withdrawal of the tubular elements (9) from the rotary element (6).

5. The device as claimed in claim 1, characterized in that it comprises means for viewing the passage of the implants which are located in the tubular element (9) aligned with the trocar (3).

6. The device as claimed in claim 1, characterized in that the means for viewing the passage of the implants comprise a window (14).

7. The device as claimed in claim 1, characterized in that the gripping means (2) have a flattened section (15).

8. The device as claimed in claim 1, characterized in that the rotary element (6) comprises a knurled wheel (16)

9. The device as claimed in claim 1, characterized in that each tubular element (9) includes means (11) for retaining the implants (10) when the device (1) is at rest.

10. The device as claimed in claim 1, characterized in that the means for retaining the implants are composed of a flexible tongue (11) arranged inside the tubular elements (9).

11. The device as claimed in claim 1, characterized in that it comprises means which retain the rotary element (6) and prevent withdrawal of the rotary element (6) once the latter has been placed in the gripping means (2).
